(19) United States
(12) Patent Application Publication
(10) Pub. No.: US 2004/0096852 A1
(43) Pub. Date: May 20, 2004

(54) Mixed fibrils

(76) Inventors: Chris Dobson, Cambridge (GB); Catherine Macphee, Cambridge (GB)

Correspondence Address: Quarles & Brady, Suite 2040, 411 East Wisconsin Avenue, Milwaukee, WI, 53202-4497, US

(21) Appl. No.: 10/432,344

(22) PCT Filed: Nov. 23, 2001
(86) PCT No.: PCT/GB01/05190

(30) Foreign Application Priority Data
Nov. 24, 2000 (GB) 0028710.2

(51) Int. Cl. C12Q 1/68; G01N 33/53; A61K 39/395; C07K 14/47; A61K 48/00; A61K 38/28

(52) U.S. Cl. 435/6; 435/7.1; 424/146.1; 514/44; 530/350; 530/303

(57) ABSTRACT

Fibrils of two or more different peptides are disclosed; these peptides can be related or unrelated. They have a variety of uses as biomaterials and nanomaterials.

### FIELD OF THE INVENTION

This invention relates to mixed fibrils.

### BACKGROUND OF THE INVENTION

The aggregation of proteins and peptides in the form of stable and highly ordered amyloid fibrils is most commonly associated with pathological conditions such as Alzheimer's disease and the transmissible spongiform encephalopathies.

The involvement of only a handful of proteins in amyloid formation in vivo has Commonly been thought of as the result of some unusual conformational characteristic of the sequences of the proteins involved. However, as disclosed in WO 00/17328, the formation of these highly ordered structures is a generic process arising from the fundamental physico-chemical properties of the polypeptide chain. At low pH, a concentrated solution of, for example, the SH3 domain of PI-3′ kinase has been shown to turn into a viscous gel after several hours. When this gel was examined by electron microscopy and other techniques it was found to contain well-defined fibrils with all the characteristics of those associated with disease-related amyloid. This protein has no connection with any known disease.

### SUMMARY OF THE INVENTION

It has now been found according to the present invention, that, typically short, peptides can be incorporated into amyloid fibrils assembled from unrelated peptides and from a full-length protein i.e. amyloid fibrils can be formed consisting of two or more distinct molecular species that co-assemble within the β-sheet array that makes up the core of these structures. Thus, for example, amyloid fibrils can be “doped” by the incorporation of peptide like molecules or ones with different side chains. Accordingly the present invention provides an amyloid fibril comprising two or more different peptides. Typically the fibril is free of other protein.

The present invention further provides:

a plastic or gel comprising mixed fibrils of the invention;

a support for the growth of cells, said support comprising mixed fibrils, a plastic or a gel of the invention;

a process for making an amyloid fibril of the invention which comprises preparing a solution of the peptides, said solution being in a state so that nucleation and fibril growth will occur over an acceptable time and allowing nucleation and fibril growth to take place;

an amyloid fibril when made by a process of the invention;

use of a fibril of the invention as a plastic or in electronics or catalysis; and

a fibril, plastic, gel or support of the invention for use in a method of treatment of the human or animal body by therapy.

### BRIEF DESCRIPTION OF THE FIGURES

FIG. 1(a) shows the fluorescence emission spectrum for F5M-TTR₁₀₋₁₉ incorporated into TTR₁₀₋₁₉ fibrils (dashed line) compared with the spectrum for free F5M-TTR₁₀₋₁₉ (solid line). FIG. 11o) shows the spectrum for dansyl-TTR₁₀₅₋₁₁₅ incorporated into TTR₁₀₅₋₁₁₅ fibrils (dashed line) compared with that for free dansyl-TTR₁₀₅₋₁₁₅ (solid line).

FIG. 2 shows the circular dichroism spectra for TTR₁₀₅₋₁₁₅ fibrils (dashed line) and TTR₁₀₅₋₁₁₅ fibrils containing 1% (w/w) F5M-TTR₁₀₋₁₉ (solid line).

FIG. 3 shows that the binding of anti-fluorescein IgG to insulin-TTR₁₀₋₁₉ mixed fibrils is periodic. FIG. 3(a) shows a histogram of the distance in nm between colloidal gold particles bound to TTR₁₀₅₋₁₁₅ fibrils containing F5M-TTR₁₀₋₁₉. FIG. 3(b) shows the a histogram of the distance between colloidal gold particles bound to insulin fibrils containing F5M-TTR₁₀₋₁₉.

FIG. 4(a) shows the conductivity of unmodified fibrils. FIG. 4(b) shows the conductivity of fibrils containing 2% w/w of fluorescently labeled peptide.

### BRIEF DESCRIPTION OF THE SEQUENCES

SEQ ID No. 1 shows the amino acid sequence of the peptide TTR₁₀₋₁₉.

SEQ ID No. 2 is the amino acid sequence of the peptide TTR₁₀₅₋₁₁₅.

### DETAILED DESCRIPTION OF THE INVENTION

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

By “protein”, as used herein, it is meant one or more proteins, protein fragments, polypeptides or peptides. The protein is any protein capable of forming fibrils and may be a pharmaceutically active protein.

We have found that amyloid fibrils consisting of two or more unrelated species can be formed. Typically, none of the peptides or proteins examined is directly implicated in any naturally-occurring disease (although such peptides and proteins are not excluded and may in particular be used in embodiments where the fibrils formed are not to be used in vivo) yet the resulting structures resemble closely those formed in vivo in pathological states. The mixed fibrils may comprise two, three, four or more different types of peptide species.

By utilizing specific conjugation chemistries, amyloid fibrils of the invention can provide a rigid scaffold that can be covalently modified by the conjugation of fully-folded and active proteinaceous enzymes or cofactors, or non-protein materials such as metals, chelating agents or optically active compounds. By showing that fibril assembly is a fundamental property of the polypeptide chain, this raises the possibility of employing these fibrillar arrays as structural building blocks in the development of novel biomaterials and nanostructures. Unlike the products of conventional organic polymerization processes, artificial proteins can be engineered with virtually absolute control of composition, chain length, sequence and stereochemical purity. The vast array of possible combinations of the 20 naturally-occurring amino acids suggests innumerable possibilities for novel biomaterials, while the introduction of the functional groups shows the ability to design nanomaterials with potentially important physical properties for a wide range of applications. Non-native systems and mimic proteins can also be introduced.

Polypeptides

In one embodiment, the proteins forming the fibril are unrelated, typically they do not have a high degree of sequence homology. Alternatively, the proteins possess the same amino acid sequence, or ones which are very similar such as homologous sequences e.g. insulin from a pig and from a human, (and are therefore related) and differ in that one possesses a pendant functional group, for example, one peptide can be labeled and the other is unlabelled. More generally, one of the proteins possess a pendant functional group and is referred to as having been functionalised.

Functional groups which can be incorporated in this way include metals, such as gold, silver, copper or heavy metals, radioisotopes, chelating groups and optically active groups including fluorescent groups such as dansyl chloride and fluorescein-5-maleimide, other proteins, drugs and biotin or magnetic particles.

By way of example two peptides, TTR₁₀₋₁₉ and TTR₁₀₅₋₁₁₅, both derived from the sequence of the human plasma protein transthyretin can be used for co-fibril formation. Each corresponds to a N-strand in the native structure of this extensively β-sheet protein. Both peptides readily assemble into fibrils with all of the structural characteristics that are diagnostic of amyloid. TTR₁₀₋₁₉ has a single cysteine residue at position 1 and this can be chemically modified with the fluorophore fluorescein-5-maleimide (F5M-TTR₁₀₋₁₉) or other suitable groups. TTR₁₀₅₋₁₁₅ does not contain any amino groups other than the terminal α-amino group; this group can be coupled to the fluorescent group dansyl chloride (dansyl-TTR₁₀₅₋₁₁₅) or other suitable groups.

In general the functional group can be attached by reaction of the peptide, if desirable after modification of a function of the peptide, with a molecule comprising the desired functional group. For example a cysteine residue, e.g. the N-terminal cysteine group, in the peptide can be reduced, for example with a phosphine such as tri-(2-carboxyethyl) phosphine, to provide a thiol group. This thiol group can be reacted with, say, fluorescein-5-maleimide. Other groups which can be functionalised include amino, carboxyl, hydroxyl and amino groups. The functionalisation can be carried out using standard reactions. Thus dansyl chloride can be reacted with an amino group.

The peptides may also be functionalised using bifunctional or multifunctional linkers. The bifunctional linkers comprise two active groups which can be used to conjugate the linker to other molecules. In the case of a hetero-bifunctional linker these groups are different and preferably such linkers are used. Typically, one of the groups of the linker is reacted with the peptide, whilst the other group is reacted with a second molecule. The linker may be conjugated to the peptide before the peptide is used to form fibrils and then the second group conjugated to the other molecule after, or during, fibril formation. The second molecule may be a peptide in another fibril or a separate polypeptide, nucleic acid or therapeutic molecule. Alternatively, the second group may be conjugated to a virus, cell or synthetic surface.

The linker may be cleavable. This may be used to release the conjugated substances from the fibril or for networks or meshes of cross-linked fibrils to be broken up or dissembled, when exposed to a suitable enzyme or chemical capable of cleaving the linker. Examples of suitable bifunctional linkers which may be used include:

APDP—(N(4-Azidosalicylamido)Butyl-3′(2′Pyridyldithio)Propionamide), cleavable, second site is non-specifically photoreactive;

ASIB ((1-(P-Azidosalicylamido)-4-(Iodoacetamido)Butane)), non-cleavable, second site is non-specifically photoreactive;

MPBH—(4-(4-N-Maleimidophenyl) Butyric Acid Hydrazide Hydrochloride) non-cleavable, second site is reactive towards carbohydrates;

PDPH—(3-(2-Pyridyldithio)Propionyl Hydrazide), cleavable, second site is reactive towards carbohydrates;

SIAB—(N-Succinimidyl(4-Iodoacetyl) Aminobenzoate) non-cleavable, second site is reactive towards primary amines, 10.6 Å spacer arm;

SMCC—(Succinimidyl-4-(N-Maleimidomethyl)), non-cleavable, second site is reactive towards primary amines, 11.6 Å spacer arm;

SMPT—(4-Succinimidyloxycarbonyl-Methyl-(2-Pyridyldithio)-Toluene), non-cleavable, second site is reactive towards primary amines, 11.2 Å spacer arm; and

SPDP—(N-Succinimidyl-3-(2-Pyridyldithio) Propionate), cleavable, second site is reactive towards primary amines, 6.8 Å spacer arm.

The hetero-bifunctional linker may be used to cross link fibrils and in a preferred embodiment a linker comprising an amine reactive NHS ester and a photoreactive arylazide is used. The linker is first joined to one of the peptide species and a suitable amount of the linked peptide introduced into fibrils. The fibrils are then subsequently exposed to UV light to cross link them and hence may be used to generate a plastic.

The peptide species comprising the fibrils may be chosen for their ability to form fibrils and/or their functional properties. In one embodiment, one of the polypeptides is chosen for its ability to form fibrils and the other for its functional properties such as its therapeutic properties or for ease of functionalisation. The structural polypeptide may be chosen so that the fibril disassociates at a particular temperature, ionic strength, or pH or when it comes into contact with a specific substance or cell type. This may mean that the rate at which the fibril disassociates may be specifically designed by varying the amount and/or type of each subunit present. This may be used to allow the controlled release of a drug at a set rate.

By choosing a structural polypeptide which results in the fibrils disassociating at a particular temperature this may allow, for example, the fibrils to disassociate when they reach body temperature. Structures made from such fibrils may be broken up by applying a raised temperature. This may enable gels of the fibrils to be liquidised or the breakdown of plastics made from the fibrils.

As well as functional groups being conjugated to the fibril polypeptides the individual peptides themselves may have activities. For example the peptides may be an agonist, an antagonist, an inhibitor or modulator such as of a receptor. The peptide may be a toxin, an enzyme, a fluorescent protein (such as green fluorescent protein), a hormone, a drug, a single chain antibody, a receptor, an epitope, or an inflammatory modulator such as an cytosine or interleukin, a cofactor, or clotting factor. In such embodiments the peptide may typically adopt its active conformation once released from the fibril but typically not before.

The fact that the fibrils are comprised of two or more types of peptides may allow pairs of drugs or therapeutic molecules to be delivered to the same location at the same time. In one embodiment this may allow both an enzyme and its substrate to be delivered to a location and the enzyme to only begin to act on a substrate once the two are released. Prior to, and during fibril formation the enzyme may be kept in an inactive state due to, for example, the conditions employed to make the fibrils.

The relative amounts of the two peptides in the fibril may also be chosen to optimize the structural and functional properties of the fibrils. The ratio of the two subunits may be altered to define the amount of therapeutic peptide released or the intensity of the fluorescence. This may allow low dose or high dose fibrils to be produced. The ratio of fluorochrome labeled, therapeutic, or functionalised peptide to the other peptide may be from 100:1 to 10:1, preferably from 50:1 to 5:1 and more preferably from 10:1 to 2:1, alternatively the amount of the other peptide may be equal or greater than the functionalised, therapeutic or labeled peptide and hence the ratio may be from 1:1 to 1:100,000, typically from 1:2 to 1:10,000, preferably from 1:5 to 1:100, more preferably from 1:10 to 1:100, but will depend on the particular use the fibril is being put to. By altering the ratio of the constituent peptides this may allow the temperature at which the fibril breaks down to be modified or to alter the rate of disassembly.

The peptide species used to form the fibrils may have different binding sites so that the fibril has several specificities. For example, the peptides may have binding or recognition sites for two different cell types and facilitate the bringing together of the two types of cells. The fibrils may also comprise binding sites to allow them to localise specifically to a particular cell type, organ or tissue. Fibrils containing a binding site for a particular substance may be used to purify or extract that substance, for example, fibrils with comprising a particular epitope may be used to affinity purify antibodies specific for the epitope.

The peptides employed in the invention are preferably synthetic. Alternatively, they may be, or be derived from, recombinant proteins. In some embodiments, they may be purified naturally occurring proteins. The fibrils may be formed in vivo such as in a non-human transgenic animal and subsequently harvested.

In addition to the 20 naturally occurring amino acids, manipulation of bacterial expression systems has allowed the incorporation of amino acids with unnatural side-chains into polypeptide chains, thereby expanding the repertoire of chemical functionalities available for exploitation. Any of these unnatural amino acids may be incorporated in the polypeptides of the invention.

Fibril Formation

The fibrils of the present invention can be obtained in a similar manner to that described in WO 00/17328 but using a solution of the different proteins, the solution being in a state so that nucleation of the protein and fibril growth will occur over an acceptable time, and allowing nucleation and fibril growth to take place.

By “nucleation”, as herein used, is meant the initiation of processes that lead to fibril formation. Fibril formation from a solution involves, successively, protein self-association, formation of aggregates and fibril growth. Thus, desirably, the initiation solution is on the verge of instability. Nucleation and growth are slow processes and conditions are normally chosen so that fibril formation occurs over a period of hours or days. It will be appreciated that if nucleation occurs too rapidly then this will often have an adverse affect on fibril formation. Fibrils for use in vivo are typically larger than aggregates.

Nucleation can be caused by a variety of means including variations in solvents, concentration, salt, ligand, temperature and pH. Thus fibrils from TTR₁₀₋₁₉ and TTR₁₀₅₋₁₁₅ can be obtained from an aqueous solution at pH about 2. Fibrils from bovine insulin can be obtained from an aqueous solution at pH about 2.5. Shaking, agitation and exposure to certain surfaces, for example the surface of a glass or plastic vessel, may cause local denaturation and thereby initiate fibril formation.

The solution comprising a protein may comprise any solvent or mixture of solvents in which nucleation can occur. For example, the solution may comprise DMSO, dioxin and/or water. Preferably the solution is an aqueous solution.

One or more organic solvents which can promote nucleation and fibril growth may be incorporated into the solution. In the case of naturally occurring proteins conditions are typically chosen to denature at least partially the protein whilst retaining conditions in which self-association can occur. The organic solvent is generally water-miscible and is preferably an alcohol or an aliphatic nitrile such as acetonitrile. The alcohol is typically a C₁₋₆ alkanol which may be substituted or unsubstituted for example by one or more halogen atoms, especially fluorine atoms. Examples include methanol, ethanol, propenyl or butanol, or fluorinated alcohols such as trifluoroethanol or hexafluoroisopropanol. Preferably the alcohol is trifluoroethanol. The concentration of alcohol is typically from 5 to 40% v/v and preferably about 25% v/v. The concentration of aliphatic nitrile can vary between wide limits and is typically from 5 to 95% v/v.

The concentration of protein in the solution is not limited in any way but it must be such that nucleation can occur. Generally the concentration is from 0.1 mM to 10 mM, preferably from 0.5 mM to 5 mM and more preferably the concentration of protein is about 1 mM.

The temperature of the solution is generally from 0° C. to 100° C. Preferably the temperature is from 0° C. to 70° C., more preferably from 0° C. to 40° C. and most preferably from 5° C. to 30° C.

The pH of the solution is any pH suitable for nucleation, typically from pH 1 to pH 10. Preferably the solution is acidic and more preferably the pH of the solution is from 1 to 6.5, for example 1.5 to 3.5.

The solution may be seeded with, for example, previously formed particles of aggregated protein; this can greatly speed up the process.

The fibrils of the present invention are suitably isolated by centrifugation, filtration or evaporation of solvent. The fibrils thus obtained may then be washed and dried and, if necessary, resuspended. The physical properties of the fibrils, such as solubility, can be altered in this way. Also the polypeptides can be tagged so that they can be removed. If a tagged mixed fibril can be formed and readily removed problems such as beta-2M aggregation can be minimised. In some cases, the functional group present in the fibrils may be used to facilitate their purification or recovery.

Where the proteins forming the fibrils are unrelated the kinetics of fibril formation are likely to be different. In such situations it has been found that the kinetics of fibril formation should be adjusted so that they are relatively evenly matched. By way of example fluorescently-labeled peptides of TTR₁₀₋₁₉ and TTR₁₀₅₋₁₁₅ can be incorporated into fibrils assembled primarily from an unrelated protein sequence, bovine insulin, which consists of two disulfide-linked chains in its largely helical native fold. The formation of fibrils from bovine insulin is well established and can readily be achieved by heating the protein to high temperatures (60° C.) at low pH e.g. 2.5. Under these conditions, however, the rate of fibril formation by the insulin molecules is significantly faster than that observed for TTR₁₀₋₁₉ and TTR₁₀₅₋₁₁₅: 7 hours for completion of the process, whereas both peptides remain soluble for 48 hours under similar conditions. The conditions of fibril formation by insulin to adjust the rate of assembly to approximately that of fibril formation by the short peptides, namely at pH 2.7 at 55° C., i.e. by adjusting, in this case reducing, the temperature and adjusting, in this case increasing the pH, the fibrils formed over a period of 10 days. In contrast the peptides alone at the concentration used for co-fibril formation (0.05 mg/ml) remained fully soluble over a period of 3 weeks under identical conditions.

The fibrils of the present invention may also be formed in vivo. For example, nucleic acid sequences encoding one, several or all of the polypeptides may be introduced into cells, such as eukaryotic and in particular mammalian cells, and expressed. In one embodiment, the sequences are introduced into the cells of an organism suffering from, or a model for, a fibril associated disease. Fibrils will then be formed comprising both the endogenous polypeptide associated with the disease and the heterologous polypeptide. This may allow the fibrils to be labelled or the targeting of cells or therapeutic substances to the fibrils. This may be useful in studying the disease state or in therapy and/or diagnosis. The organism may be a non-human transgenic animal into which the heterologous sequences have been introduced. Alternatively it may be an organism into which transformed cells are introduced or a vector carrying the sequences is introduced.

Typically, the nucleic acid sequences are targeted to, or comprise sequences which ensure the expression in, the same cell type which is synthesizing the endogenous peptide. In addition, their expression may be inducible rather than constitutive.

In cases where one of the peptides is labeled, incorporation into a fibril may result in a change in the properties of the label. For example, the label may be a fluorochrome and incorporation into the fibril of the labeled peptide may result in a change its emission spectra. Such changes may be used to monitor fibril formation and breakdown both in vitro and in vivo.

In some embodiments, by varying the type of peptide species used and/or their relative amounts the shape of the fibril and the periodicity of the doped peptide may be altered. This may allow peptides with a particular shape, such as a helical twist or a kink, which are hollow or ribbon shaped to be produced or to set the distance between functional groups.

Formed Structures, Gels, and Plastics

Non-native self-assembling peptide polymers have previously been synthesized, following careful design principles based on side-chain interactions, to form reversible and pH-dependent hydrogels, O-sheet peptide nanotubes that can act as non-specific pores within membranes, P-sheet “nanotapes” and lamellar crystals. The formation of amyloid fibrils of the present invention typically requires no explicit design process since it is a fundamental property of the polypeptide backbone. The solubility, stability and conformational properties of monomeric precursors will, however, vary with the specific sequence allowing exploitation of a wide range of environments for fibril assembly. The side-chain interactions within β-sheets of different compositions will furthermore influence and modulate the physical properties and the morphologies of the resulting fibrils. The formation of amyloid fibrils as tubular structures with a hollow core, flat ribbon-like morphologies, twisted fibrils where the periodicity of the twist is dictated by the identity of the protein or peptide precursor and these can be chosen to tailor the fibril to its specific use.

The identity of the protein precursor influences the average diameter and homogeneity of the resulting fibril population. Fibril assembly can be reversed on modulation of the solvent environment the fibrils of this invention can find utility as chemically reversible and biodegradable gels. Indeed, it seems likely that the amyloid fibrils from native proteins and peptides are closely related to some of these artificially designed systems. This is, therefore, a valuable indication that nanostructures and devices could be made if desired.

The fibrils of the inventions may be cross-linked, typically by using bifunctional linkers and preferably a hetero-bifunctional linker. In particular, one of the peptides used to form the fibrils may be reacted with a linker, then incorporated into a fibril, and the fibrils subsequently cross-linked using the second of the linker's functional groups. For example, a bifunctional linker comprising an amine reactive NHS ester and a photoreactive arylazide may be used with the peptide of choice being conjugated to the linker via the free amino group and kept in the dark. The modified peptide can then be incorporated into fibrils and afterwards the fibrils cross-linked by exposure to near UV light to activate the photoreactive arylaride.

Fibril cross-linking can be used to produce a stronger, more robust network or mesh of fibrils. The cross-lining can, in effect, produce a plastic. The degree of doping can be adjusted to alter the amount of cross-linking and hence the strength of the plastic produced. The plastic may be formed in a particular shape, such as a tubular structure.

The plastics can be used in a number of devices or structures and may also be used to coat or cover devices. For example, the plastics may be used to form or cover a stent, valve, patch or support. They may be used to cover wounds or in bandages or plasters.

The plastics may be used as a support on which cells can be grown on and the whole structure or the cells can then be used as a graft or transplant. For example, the plastic may be used to grow skin cells on, or islets cells. The linker may also include a cleavage site to allow the cross-linked mesh to be broken down. This may be used, to remove the plastic whilst leaving the cells intact. This may be done in vitro or in vivo and in particular in vivo. The cleavage may be carried out chemically or enzymatically and is typically done in such a way as to cause minimal damage to the surrounding tissues. This may allow shaped arrangements of cells to be produced such as in a pouch, bladder or vessel.

Peptide-DNA Fibrils

The peptides of the present invention may be conjugated to nucleic acids or nucleic acid analogues such as DNA, RNA or PNA. Typically, a bifunctional linker is used to link the nucleic acid to the fibrils. The nucleic acid molecule may be modified to include a group through which it can be conjugated or bound to a peptide either directly or through a linker. Preferably, the nucleic acid may posses a free sulfhydryl group and be conjugated to the peptide through that. The peptide may be conjugated with the nucleic acid before, after or during fibril formation, but preferably before. The nucleic acid may be joined to the fibril through a cleavable linker so that it can be subsequently released.

The nucleic acid molecules may be either double or single stranded and may range in size from a short oligonucleotide to considerably longer, such as from 10 to 5,000 bases, preferably from 20 to 1,000 bases, more preferably from 25 to 50 bases in length. The peptide-nucleic acid may be immobilized on a surface such as a membrane or in silico and may be in the form of a micro-array or chip. These may be used to detect the presence and quantity of a specific target sequence in a test solution. This may be employed in diagnosis or genotyping.

The mixed fibrils conjugated with DNA or other nucleic acids may also be used as a delivery system for the nucleic acid into an organism for gene therapy purposes. The nucleic acid may drive the expression of a protein or alternatively may be an antisense molecule employed to block the expression of a target gene. The nucleic acid may be a catalytic nucleic acid.

Conductivity

The fibrils of the invention may display conductivity and in particular those comprising fluorescent functional groups may do so, and display particularly good conductivity. The ability to carry current may be important in the use of the fibrils in nanotechnology and biocomputers. In one embodiment hollow fibrils may be used.

The fibrils conductance may change according to the surrounding conditions and/or the binding of target molecules. This will also allow the use of the fibrils in sensors or determine the presence or amount of a particular analyte. The fibrils are typically linked to a means for detecting the change in conductivity on binding.

Therapeutic Uses of Mixed Fibrils

The fibrils may be used to treat diseases associated with amyloid deposition. Examples of such diseases which may be treated, prevented or ameliorated include diabetes, senility and dementia and in particular Alzheimer's and Parkinson's. Prion disorders such as Creutzfield-Jakob disease (CJD), BSE, scrapie, fatal familial insomnia (FFI), and kuru may be also treated and in particular new variant CJD. Diabetes may also be treated and in particular late onset diabetes.

It has been demonstrated that amyloid protein A (AA) amyloidosis in mice can be accelerated by the injection of preformed amyloid fibrils assembled from a number of peptide sources, including residues 20-29 of islet amyloid polypeptide (APP) and the sequences TTR₁₀₋₁₉ and TTR₁₀₅₋₁₁₅ Thus injection of fibrillar material can provide a seeding mechanism for the deposition of amyloid protein A. The ability to incorporate a different polypeptide and hence to form a mixed fibril in vivo could be used to block fibril formation, to incorporate a drug or introduce a drug binding site. It could also be used as a diagnostic or therapy for the fibril associated condition.

The mixed fibrils may be used to deliver drugs to individuals suffering from non-fibril associated diseases such as cardiovascular disorder or a cancer such as a soft tissue cancer, sarcoma, leukemia or lymphoma. The particular fibril employed may be chosen so that its characteristics such as the rate it disassembles, the temperature, pH or location it disassembles at, or the dosage and combination of drugs it releases are tailored to the needs of the patient. The fibrils may also be used to facilitate the bringing together of two entities such as different types of cells or a tissue and a cell due to the presence of bringing sites for each cell. Labeled fibrils may also be used in imaging.

The fibrils may be formulated with standard pharmaceutically acceptable carriers and/or excipients as is routine in the pharmaceutical art. For example, a suitable substance may be dissolved in physiological saline or water for injections. The exact nature of a formulation ill depend upon several factors including the particular fibrils to be administered and the desired route of administration. Suitable types of formulation are fully described in Remington's Pharmaceutical Sciences, Mack Publishing Company, Eastern Pennsylvania, 17^{th} Ed. 1985, the disclosure of which is included herein of its entirety by way of reference.

The fibrils may be administered by enteral or parenteral routes such as via oral, buccal, anal, pulmonary, intravenous, intra-arterial, intramuscular, intraperitoneal, topical or other appropriate administration routes.

A therapeutically effective amount of the fibrils is administered to a patient. The dose of the fibril may be determined according to various parameters, especially according to the substance used; the age, weight and condition of the patient to be treated; the route of administration; and the required regimen. A physician will be able to determine the required route of administration and dosage for any particular patient. A typical daily dose is from about 0.1 to 50 mg per kg of body weight, according to the activity of the specific modulator, the age, weight and conditions of the subject to be treated, the type and severity of the degeneration and the frequency and route of administration. Preferably, daily dosage levels are from 5 mg to 2 g.

The fibrils of the invention may also be used to form gels to encapsulate drugs or to make gels impregnated or carrying a drug. The gel typically breaks down to release the drug either because the capsule is broken or the impregnated drug released. The breakdown of the gel is typically the result of the disassembly of the fibrils. The breakdown may be made temperature or pH dependent and may occur slowly or rapidly to ensure the desired release rate of the drug. Such gels may be used to produce pills or capsules or alternatively implants. The gels may also be used to coat devices introduced into the body such as stems or patches to slowly release the drug at that site.

### EXAMPLES

The following Examples further illustrate the present invention. Peptide synthesis. TTR₁₀₋₁₉ (SEQ ID No:1—CPLMVKVLDA) and TTR₁₀₅₋₁₁₅ (SEQ ID No:2—YTIAALLSPYS) were synthesized on an Applied Biosystems 430A automated peptide synthesizer using standard Fmoc chemistry, within the Oxford Centre for Molecular Sciences. After cleavage from the support, the peptides were purified by RP-HPLC on a Beckman System Gold HPLC using either an analytical Brownlee 4.6×30 mm C8 reverse-phase column or a preparative Vydac 10×250 mm C-18 reverse phase column. Fractions eluting from the column were analysed by MALDI-TOF mass spectrometry, and fractions of the desired molecular weight pooled and lyophilised.

Peptide Labeling

TTR₁₀₋₁₉ labeling with fluorescein. The N-terminal cysteine residue of TTR₁₀₋₁₉ was the target for labeling by the thiol-reactive fluorophore fluorescein-5-maleimide (F5M; Molecular Probes, Eugene, Oreg.). TTR₁₀₋₁₉ was resuspended at 1 mg/ml in H₂O, and the cysteine residue reduced by the addition of a 10-fold molar excess of tris-(2-carboxyethyl) phosphine (TCEP; Molecular Probes, Eugene, Oreg.) and incubation for 1 h at room temperature. F5M in DMSO was added to the peptide solution to give a 10-fold molar excess of fluorophore over peptide in a final concentration of 10% DMSO. Under these conditions TTR₁₀₋₁₉ was fully soluble. The mixture was incubated overnight at 4° C. to ensure maximum labeling. The labeling mixture was then applied to a disposable SepPak C-18 reverse-phase chromatography column (Waters, Milford, Mass.) and washed with H₂O/CH₃CN (90:10 v/v). TCEP and unreacted label eluted in the wash. The peptide was then eluted from the column with H₂O/CH₃CN (40:60 v/v), and a mixture of labeled and unlabelled peptide was detected by MALDI-TOF mass spectrometry. The labeling efficiency was estimated at −70% by mass spectrometry, and also by absorption measurements assuming an extinction coefficient of 83,000 at 492 nm.

TTR₁₀₅₋₁₁₅ labeling with dansyl chloride. The N-terminal amino group of TTR₁₀₅₋₁₁₅ was the target for labeling with dansyl chloride (Molecular Probes, Eugene, Oreg.). TTR₁₀₅₋₁₁₅ was resuspended at 1 mg/ml in H₂O/CH₃CN (80:20 v/v). Dansyl chloride in DMSO was added to the peptide solution to give a 10-fold molar excess of fluorophore over peptide in a final concentration of 10% DMSO. The pH was adjusted manually to 9.0 with the addition of ammonia. Under these conditions TTR₁₀₅₋₁₁₅ was fully soluble. The mixture was incubated for 2 h at room temperature to ensure maximum labeling. The labeling mixture was then applied to a disposable SepPak C-18 reversed phase chromatography column (Waters, Milford, Mass.) and washed with H₂O. There was no detectable elution of material during the wash step. The peptide was then eluted from the column with H₂O/CH₃CN (50:50 v/v), and a mixture of labeled and unlabelled peptide was detected by MALDI-TOF mass spectrometry. Unreacted dansyl chloride did not elute from the column under these conditions. The labeling efficiency was estimated at −80% by mass spectrometry, and also by absorption measurements assuming an extinction coefficient of −4000 at 370 nm.

Incorporation of labeled peptides into ‘self’ fibrils. To investigate whether chemical modification of the peptides interfered with the peptide self-assembly, we first formed amyloid fibrils containing a mixture of labeled and unlabelled peptides of the same amino acid sequence. After incubation for 7 days the resulting fibrils were examined by ultracentrifugation, electron microscopy (EM), circular dichroism (CD) spectroscopy and fluorescence methods (see below).

Fibril formation. TTR₁₀₋₁₉ fibrils, in the presence and absence of F5M-TTR₁₀₋₁₅, were generated by resuspending the peptide at 10 mg/ml in H₂O at pH 2.0 and incubating at room temperature for 5 days. In fibrils containing the labeled species, the molar ratio of unlabelled to labeled peptide was 100:1.

TTR₁₀₅₋₁₁₅ fibrils, in the presence and absence of dansyl-TTR₁₀₅₁₁₅, were generated by resuspending the peptide at 10 mg/ml in CH₃CN/H₂O (20:80 v/v) at pH 2 and incubating at 37° C. for 7 days. In fibrils containing the labeled species, the molar ratio of unlabelled to labeled peptide was 100:1.

Bovine insulin (Sigma Chemical Co., MO) fibrils were generated by resuspending the protein at 11.5 mg/ml (2 mM) in H₂O at pH 2.5 and incubating the suspension at 70° C. for 1 h. The protein solution was plunged into liquid N₂, and then returned to 70° C. for a further 4 h.

Mixed fibril formation. Both TTR₁₀₋₁₉ fibrils containing dansyl-TTR₁₀₅₋₁₁₅ and TTR₁₀₅₋₁₁₅ fibrils containing F5M-TTR₁₀₋₁₉ were prepared at a molar ratio of unlabelled to labeled peptide of 100:1. The stock solution containing a total of 10 mg/ml peptide was prepared in CH₃CN/H₂O (10:1 v/v) adjusted to pH 2 with H₂O and incubated at 37° C. for 10 days. Bovine insulin was prepared as a stock solution of 9.7 mg/ml in H₂O at pH 2.7, containing a molar ratio of insulin to labeled peptide species (dansyl-TTR₁₀₅₋₁₁₅ or F5M-TTR₁₀₋₁₉) of 40:1. The solution was incubated at 55° C. for 10 days. Fibril formation was assessed by CD spectroscopy and by EM.

Comparison of CD spectra acquired over the 10-day incubation period indicates the transition from the predominantly α-helical structure of bovine insulin to the β-sheet structure typical of amyloid fibril formation. The resulting fibrils display a characteristic helical twist along the long axis of the fibril, repeating every 60 nm as determined by electron microscopy. Approximately 60% of the F5M-TTR₁₀₋₁₉ sedimented with the fibrils on ultracentrifugation, and the fluorescence properties of the probe that colocalised with the fibrils were consistent with insertion of the fluorescein group into the fibril structure (6 nm spectral shift, anisotropy=0.16; Table 1). The fluorescence properties of purified cofibrils containing dansyl-TTR₁₀₅₋₁₁₅ were also consistent with insertion of the fluorophore into the insulin fibrils, with a 32 nm blue shift in the wavelength of maximum emission to 470 nm, and an increase in anisotropy to 0.19 (Table 1). More than 70% of the peptide was found in the pellet on centrifugation of the fibrils.

Circular dichroism spectroscopy. CD spectra were acquired on a Jasco model J-720 spectropolarimeter using a 0.1 cm pathlength quartz cuvette. The temperature was kept constant at 25° C. by use of a circulating water bath. Each spectrum represents the average of 4 scans acquired at 0.5 nm intervals between 200-250 nm, with a response time of 4 s. The data are expressed in millidegrees rather than mean residue ellipticity due to the difficulty associated with accurately determining the absolute concentration of an aggregated peptide. For this reason, it was not possible to accurately determine the amount of P-sheet structure within the complexes. Furthermore, in all cases the absorption of the sample was monitored concurrently to the circular dichroism signal, to avoid possible distortion of the spectrum due to light scattering or absorption flattening phenomena. FIG. 2 shows the circular dichroism spectroscopy of TTR₁₀₅₋₁₁₅ fibrils in the presence and absence of 1% (w/w) F5M-TTR₁₀₋₁₉. TTR₁₀₅₋₁₁₅ fibrils (dashed line) were generated by resuspending the peptide at 10 mg/ml in 20% CH₃CN at pH 2 and incubating for 24 hours at 37° C., followed by 7 days incubation at room temperature. Fibrils containing 1% (w/w) F5M-TTR₁₀₋₁₉ (solid line) the 10 mg/ml peptide stock solution was incubated for 10 days at 37° C. in solution containing 10% CH₃CN. This shows the formation of extensive 13-sheet structure.

Electron microscopy. Electron micrographs of amyloid fibrils were acquired on a Jeol JEM-1010 transmission electron microscope at 80 kV excitation voltage. 2 μl of peptide at a concentration of 10-60 μg/ml was applied to formvar- and carbon-coated copper grids. The fibrils were washed by the successive addition of three 10 μl aliquots of water, each wash step followed by drying with filter paper. The fibrils were stained with by the addition of 10 μl uranyl acetate (1% w/v), and immediately dried with filter paper.

Electron micrographs of:

TTR₁₀₅₋₁₁₅ fibrils formed by incubation for 24 hours at 37° C., followed by 7 days incubation at room temperature in 20% CH₃CN pH 2;

cofibrils consisting of TTR₁₀₅₋₁₁₅ containing 1% (w/w) F5M-TTR₁₀₋₁₉, formed by incubation for 10 days at 37° C.

were obtained and studied and found to show that the labeled fibrils formed are visually very similar to those formed by the unlabelled fibrils.

Fluorescence Spectroscopy. Fluorescence spectroscopic measurements were performed on a Perkin-Elmer LS50B luminescence spectrometer equipped with a fast filter accessory to enable anisotropy measurements. Quartz cuvettes with a 1 cm pathlength were employed throughout. The temperature was kept constant at 25° C. by use of a circulating water bath. Slit widths were 2.5 nm and 10 nm unless otherwise stated. Samples containing fluorescein were excited at 490 nm, with emission detected between 500-600 nm. Samples containing dansyl chloride were excited at 370 nm, and the emission measured between 400-550 nm. The absorbence of each sample at the excitation wavelength was also monitored using a Cary-3 absorbence spectrometer, to avoid scattering artefacts and inner filtering effects. All anisotropy measurements were monitored over 10 sec to rule out photobleaching phenomena and then averaged.

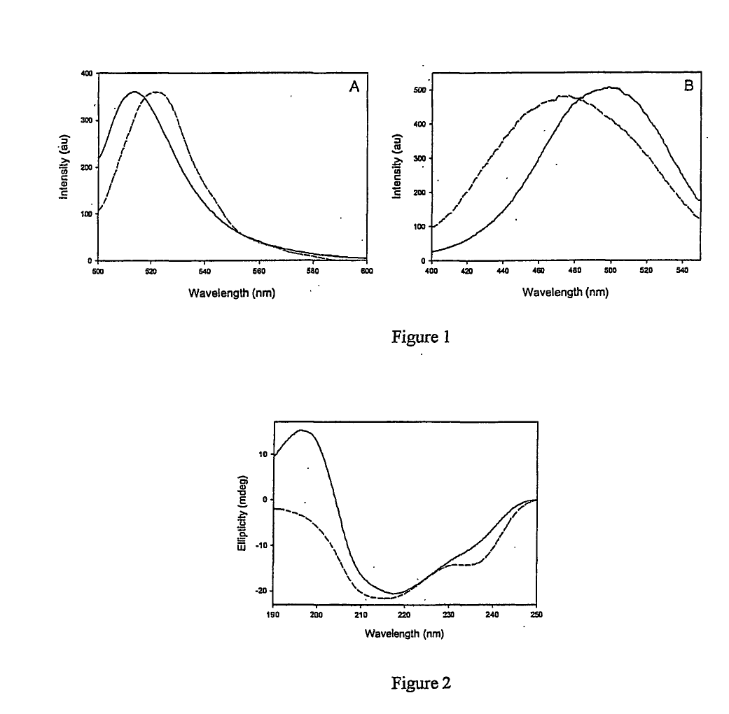

Sedimentation of the fibrils by ultracentrifugation resulted in partitioning of the fluorescent peptides almost exclusively into the fibrillar fraction, with less than 5% remaining in the supernatant. FIG. 1 shows fluorescence emission spectra of labeled peptide fibrils. Panel a: F5M-TTR₁₀₋₁₉ incorporated into TTR₁₀₋₁₉ fibrils (dashed line) is compared with F5M-TTR₁₀₋₁₉ (solid line) in 20 mM sodium phosphate buffer, pH 1.0 at a final labeled peptide concentration of 0.3 μM. Panel b: dansyl-TTR₁₀₅₋₁₁₅ incorporated into TTR₁₀₅₋₁₁₅ fibrils (dashed line) is compared with dansyl-TTR₁₀₅₋₁₁₅ (solid line) in water at a final labeled peptide concentration of 0.9 μM. The fluorescence spectra have been normalised. As shown in FIG. 1A, the fluorescence emission of the fluorescein moiety of F5M-TTR₁₀₋₁₉ undergoes a red-shift of 9 nm when incorporated into TTR₁₀₋₁₉ fibrils, and the anisotropy of the fluorescein increases from 0.03 for the monomeric peptide in aqueous solution to 0.17 on formation of the fibrils (see Table 1). This increase in the anisotropy of the fluorescent moiety indicates incorporation of the fluorophore into large complexes that exhibit slow rotational motion. The dansyl moiety of dansyl-TTR₁₀₅₋₁₁₅ exhibits a blue-shift of 26 nm when incorporated into fibrils comprising TTR₁₀₅₋₁₁₅ (FIG. 1B) and an increase in the fluorescence anisotropy from 0.02 to 0.13 (Table 1). Fibrils were pelleted by centrifugation at 9500 g for 15 min, the pellet resuspended in H₂O pH 2 and deposited on a glass slide and imaged using fluorescence light microscopy. These spectral changes provide a means of monitoring the incorporation of each of the two labeled peptides into fibrils composed predominantly of the other peptide. TTR₁₀₋₁₉ containing 1% (w/w) dansyl-TTR₁₀₅₋₁₁₅ was resuspended at a final total peptide concentration of 10 mg/ml in 10% CH₃CN adjusted to pH 2 with TFA. The sample was incubated at 37° C. for 10 days after which time the CD spectrum indicated the formation of significant O-sheet structure, and fibrils were observed by EM. The fluorescence spectrum shows a significant blue shift in the emission from the dansyl moiety when compared to soluble peptide (Table 1), and the anisotropy of the probe increases from 0.03 to 0.17. Greater than 90% of the labeled peptide partitioned with the fibrils on sedimentation by ultracentrifugation.

The fibrils were pelleted by centrifugation at 9500 g for 15 min, the pellet resuspended in H₂O pH 2 and deposited on a glass slide and imaged using fluorescence light microscopy. Fluorescence light microscopy of this pelleted material demonstrated a green fluorescence, consistent with insertion of the probe into high molecular weight structures. Similarly, 10 mg/ml TTR₁₀₅₋₁₁₅ containing only 1% by weight of F5M-TTR₁₀₋₁₉ incubated for 10 days at 37° C. in 10% CH₃CN formed extensive 13-sheet structure as indicated by CD. The shape of the CD spectrum is, however, substantially different to that of TTR₁₀₅₋₁₁₅ alone: a prominent negative band previously observed at ˜235 nm, which we ascribe to the formation of a turn structure in the assembled fibrils, decreases in magnitude (FIG. 2). This suggests a substantial change in the secondary structure of the peptides in the fibrillar array. The fibrils observed by EM are different in appearance from the almost crystalline structures formed by TTR₁₀₅₋₁₁₅ alone, and we observe cofibrils with a regular periodic twist in the electron micrographs. Fluorescence spectroscopy indicates little change in the spectral properties of the fluorescein moiety, which exhibits a red shift of only ˜2 nm, but the anisotropy increases substantially to 0.22. These experiments together indicate that it is possible to generate cofibrils of two different peptides, strongly suggesting that the identity of the amino acid sidechains is not critical for the ability of a polypeptide chain to form fibrils.

In order to establish the location of the fluorescent peptides doped into the fibril assemblies, we developed an immmunogold labeling procedure. The fluorescein moiety provides a ready antigenic target for antibody binding, and, on addition of a second antibody labeled with colloidal gold, can be detected by electron microscopy. This technique is similar in concept to that employed for identification of pathological proteins, by way of in situ staining of thin tissue sections.

TTR₁₀₋₁₉ fibrils containing 1% by weight of F5M-TTR₁₀₋₁₉, were probed with rabbit anti-fluorescein IgG, which was then probed with gold-labeled goat anti-rabbit IgG antibody (scale bar: 200 nm). The 5 nm gold particles were observed to be associated with the various fibrils, whereas TTR₁₀₋₁₉ fibrils alone did not elicit a reaction to the antibody. Measurement of the distances between successive gold clusters bound to the fibril indicates that the gold particles are distributed on average every 40-50 nm, but at irregular intervals along the fibril length. The hydrogen-bonding distance between peptides along the long axis of the fibril is 4.7 Å, so the average distance between clusters suggests that the antigenic label is incorporated every 80-100 peptides. During fibrillogenesis the fluorescently labeled peptide, which makes up approximately one in every hundred of the peptide precursors, appears to be incorporated randomly into the growing fibril. Immunogold labeling of cofibrils made up of TTR₁₀₅₋₁₁₅ containing a trace (1% w/w) of F5M-TTR₁₀₋₁₉ shows gold particles are bound at approximately 40-60 nm intervals along the length of the fibril, again suggesting random insertion of the guest peptide into the growing fibril 1-sheet scaffold. A further control experiment in which labeled peptide was added to preformed TTR₁₀₅₋₁₁₅ fibrils did not elicit antibody binding, indicating that F5M-TTR₁₀₋₁₉ does not merely bind non-specifically to fully-assembled fibrils.

Immunogold labeling. 2 μl of peptide at a concentration of 10-60 μg/ml was applied to formvar- and carbon-coated grids. After 1 min the solution was blotted with filter paper, and the grids blocked with Hanks' balanced salt solution (ABSS; Sigma Chemical Co, Mo.) containing 1% (w/v) ovalbumin for 5 min. Sample treatment was performed by inverting the grids onto a 20 μl droplet of the appropriate solution placed on a sheet of laboratory parafilm. The grids were incubated with the primary antibody (rabbit anti-fluorescein IgG diluted 1/10 into HBSS, Molecular Probes) for 30 min at room temperature before washing 3 times for 5 min with the blocking solution (BSS/ovalbumin). The secondary antibody, colloidal gold-labeled goat anti-rabbit IgG (Sigma Chemical Co) was diluted 1/10 in HBSS, and applied to the grids for 30 min. The grids were washed three times for 10 min in blocking solution, fixed in 0.5% (v/v) glutaraldehyde for 10 min, washed with H₂O for 1 min and stained with 0.5% (w/v) uranyl acetate.

Insulin fibrils containing F5M-TTR₁₀₋₁₉, were negatively stained with uranyl acetate and electron micrographs taken, these showed the regular nature of the helical twist along the length of the fibril. Immunogold labelling of the bovine insulin fibrils containing 1% (w/w) F5M-TTR₁₀₋₁₉ showed gold clusters associated with the fibrils, indicating the incorporation of the labeled peptide, and there was no non-specific binding of F5M-TTR₁₀₋₁₉ on incubation of soluble peptide with preformed insulin fibrils.

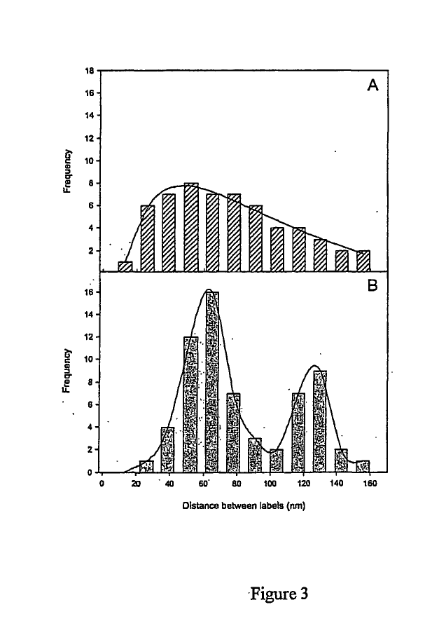

FIG. 3 shows that the binding of anti-fluorescein IgG to insulin-TTR₁₀₋₁₉ cofibrils is periodic. Panel a: Histogram showing the distance in nm between colloidal gold particles bound to TTR₁₀₅₋₁₁₅ fibrils containing F5M-TTR₁₀₋₁₉ from the immunogold labeling images obtained above. Panel b: listogram showing the distance in nm between colloidal gold particles bound to insulin fibrils containing F5M-TTR₁₀₋₁₉ from the immunogold labeling images obtained above. Unlike the cofibrils prepared with TTR₁₀₋₁₉ or TTR₁₀₅₋₁₁₅ (FIG. 3a), the binding of gold-labeled antibody to insulin-TTR cofibrils appears to exhibit a degree of regularity. The periodicity is presented as a histogram in FIG. 3b, which indicates that the labeling occurs at ˜60 and ˜125 nm intervals. A possible interpretation of this result is the regular insertion of the peptide into the growing insulin fibrils: the helical twisting of insulin fibrils every 60 nm along the fibre axis suggests at least one structural phenomenon that repeats at regular intervals. The periodic binding of gold particles may represent insertion of the peptide into the growing insulin fibril only on fibril twisting. However current models of fibril assembly indicate that protofilament substructures are formed first, which then associate and adopt a helical pitch only in the assembled fibril. We therefore believe that the observed peridocity is due to the exposure of the antigen to the aqueous phase at intervals defined by the twisting of the fibril, and that peptide incorporation into the growing protein fibril once again occurs randomly.

It is believed, although this does not form part of this invention, that the fact that unrelated peptides can be incorporated into amyloid fibrils provides compelling support for a generic structure of these fibrils that is dictated not by sidechain-specific interactions but by the formation of a network of hydrogen bonds between the invariant groups that make up the polypeptide backbone. In correctly folded globular proteins, the backbone is involved in a series of stabilising interactions and thus amyloid fibril formation is observed only following partial denaturation or destabilisation of a stable tertiary structure or the physiologically inappropriate generation of unstructured peptide fragments in vivo. The identity of the amino acid sidechains will affect the structure and stability of the protein precursor, but will not dictate the fundamental O-sheet hydrogen-bonding network of the assembled fibril. Different-sidechains will pack together in different ways within the assembled sheet structure, as indeed is observed in globular structures. It is likely that for some sequences particularly favorable contacts will be made between different sidechains and result in more favorable packing interactions. Such sequences may therefore show high propensities to form fibrillar structures. But these propensities, and the stability of the fibrils under specific solution conditions, will also depend on the intrinsic properties of the soluble form of the polypeptide chain, such as its interaction with solvent and the stability of the globular structure. Moreover, the identity of the amino acid sidechains will also influence the interactions between the unstructured regions that are thought to have an effect on the self-assembly of protofilaments into amyloid fibrils. In this way, different protein species may give rise to amyloid fibrils with differences in the detailed morphology, but with a common underlying hydrogen-bonding β-sheet network. Thus the present invention has wide applicability.

Fibril cross-linking. Peptide was modified by the addition of a hetero-bifunctional cross-linker consisting of an amine reactive NHS ester and a photoreactive arylazide in the dark. The cross-linker conjugates to the peptide via free amino groups, leaving the reactive arylazide, leaving the reactive arylazide. About 10% w/w of the peptide was incorporated into amyloid fibrils as described above and then kept in the dark. The mature fibrils were then exposed to near UV light which resulted in aligned and cross-linked sheets of fibrils which chemically resembled naturally occurring plastics. Electron micrographs of the fibrils before UV cross-linking, stored in the dark and after UV cross-linking confirmed that a dense cross-linked network resulted from the exposure to UV light.

Peptide-DNA mixed fibrils. Single stranded DNA was chemically conjugated to a peptide by exploiting thiol chemistry. 1% of the peptide was subsequently introduced into amyloid fibrils comprising predominately the same peptide but without the conjugated DNA. The successful introduction of the DNA conjugated peptides into the fibrils was demonstrated by immunogold labeling. Transmission electron micrographs for amyloid fibrils including the DNA conjugated peptide, negatively stained with uranyl acetate and immunogold labeling demonstrated the successful incorporation of the DNA-peptide subunits into the fibrils.

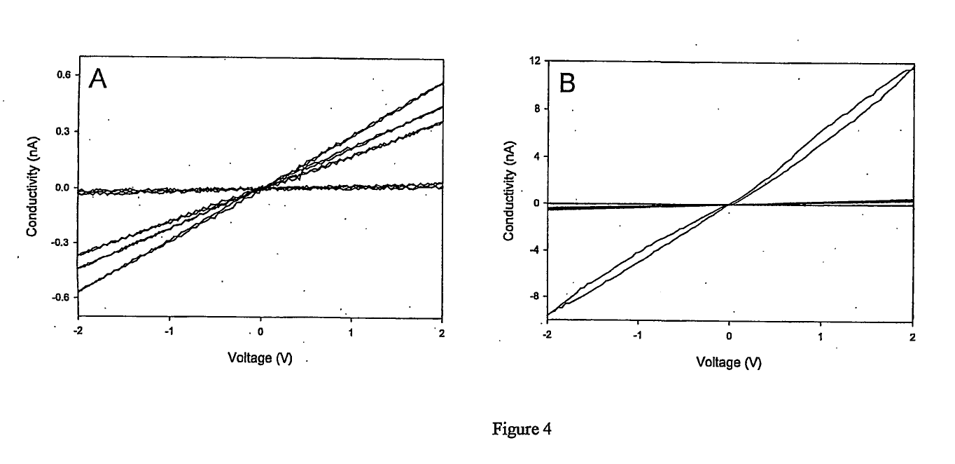

Conductivity measurements. The conductivity of several of the fibrils formed above was measured. The fibrils were deposited on mica and allowed to dry under air. Silver paste electrodes were deposited onto the fibril film. Many of the fibrils displayed conductivity. In particular, the fluorescently labeled peptides displayed high conductivity, typically about 10 times that of unmodified fibrils. FIG. 4(a) shows the conductivity of unmodified fibrils. FIG. 4(b) shows the conductivity of fibrils containing 2% w/w of fluorescently labeled peptide.

TABLE 1
The Spectral Properties of Fluorescently-Labeled Amyloid Fibrils

| Fluorophore | Fibrils | λₘₐₓ^{a}(nm) | Δλₘₐₓ^{b} | anisotropy^{c} |
|---|---|---|---|---|
| F5M-TTR₁₀₋₁₉ | — | 518 | — | 0.03 |
| F5M-TTR₁₀₋₁₉ | TTR₁₀₋₁₉ | 527 | 9 | 0.17 |
| F5M-TTR₁₀₋₁₉ | TTR₁₀₅₋₁₁₅ | 520 | 2 | 0.22 |
| F5M-TTR₁₀₋₁₉ | Insulin | 524 | 6 | 0.16 |
| TTR₁₀₅₋₁₁₅-dansyl | — | 502 | — | 0.02 |
| TTR₁₀₅₋₁₁₅-dansyl | TTR₁₀₋₁₉ | 476 | 26 | 0.16 |
| TTR₁₀₅₋₁₁₅-dansyl | TTR₁₀₅₋₁₁₅ | 472 | 30 | 0.13 |
| TTR₁₀₅₋₁₁₅-dansyl | Insulin | 470 | 32 | 0.19 |

^{a}The wavelength of maximum fluorescence emission
^{b}The change in the wavelength of maximum fluorescence emission on incorporation of the labeled peptide into fibrils
^{c}The fluorescence anisotropy, acquired atₘₐₓ

1. An amyloid fibril comprising two or more different peptides.

2. A fibril according to claim 1 in which the peptides are unrelated.

3. A fibril according to claim 1 in which the peptides have the same amino acid sequence, but wherein one or more of the peptides is modified.

4. A fibril according to any one of the preceding claims wherein at least one of the peptides possesses a pendant functional group.

5. A fibril according to claim 4 in which the functional group comprises a metal, a chelating group, an optically active group, a protein, a drug, an antibody, a linker and/or a nucleic acid.

6. A fibril according to claim 5 in which the optically active group is a fluorescent group.

7. A fibril according to claim 5 in which the fluorescent group is dansyl chloride or fluorescein-5-maleimide.

8. A fibril according to claim 5, wherein the nucleic acid is single stranded DNA.

9. A fibril according to any one of the preceding claims, wherein one or more of the peptides is a therapeutic molecule.

10. A fibril according to claim 11, wherein the therapeutic molecule is an agonist, inhibitor, antagonist, single chain antibody, inflammatory modulator or enzyme.

11. A fibril according to any one of the preceding claims in which at least one of the peptides does not occur naturally.

12. A fibril according to any one of the preceding claims in which the different peptides are present randomly in the fibril.

13. A fibril according to any one of the preceding claims 1 to 11 in which one peptide occurs at regular intervals along the length of the fibril.

14. A fibril according to any one of the preceding claims comprising a long peptide and a short peptide.

15. A fibril according to any one of the preceding claims which possesses at least one of the following peptides: TTR₁₀₋₁₉, TTR₁₀₅₋₁₁₅ and bovine insulin.

16. A plastic or gel comprising mixed fibrils as defined in any one of the preceding claims.

17. A plastic according to claim 16, wherein the mixed fibrils are cross-linked.

18. A support for the growth of cells, said support comprising mixed fibrils according to any one of claims 1 to 15 or a plastic or gel according to claim 16 or 17.

19. A process for making an amyloid fibril as claimed in any one of the preceding claims which comprises preparing a solution of the peptides, said solution being in a state so that nucleation and fibril growth will occur over an acceptable time and allowing nucleation and fibril growth to take place.

20. A process according to claim 19 in which the conditions for fibril formation are adjusted so that the kinetics of the protein species is approximately the same.

21. A process according to claim 19 or 20, which additionally comprises conjugating one or more of the peptides to a bifunctional linker.

22. A process according to claim 21, which also comprises cross linking the fibrils or using the linker to conjugate nucleic acids to the peptide.

23. An amyloid fibril when made by a process as claimed in any one of claims 19 to 22.

24. Use of a fibril as claimed in any one of claims 1 to 15 as a plastic or in electronics or catalysis.

25. A fibril as claimed in any one of claims 1 to 15, plastic or gel according to claim 16 or 17 or a support according to claim 18 for use in a method of treatment of the human or animal body by therapy.


SEQUENCE LISTING (2 sequences)

SEQ ID NO: 1 - protein, 10 aa, Artificial sequence
  Synthetic peptide
CPLMVKVLDA

SEQ ID NO: 2 - protein, 11 aa, Artificial sequence
  Synthetic peptide
YTIAALLSPYS